(19) United States
(12) Patent Application Publication
(10) Pub. No.: US 2026/0002879 A1
(43) Pub. Date: Jan. 1, 2026

(54) SENSOR APPARATUS

(71) Applicant: emz-Hanauer GmbH & Co. KGaA, Nabburg (DE)

(72) Inventors: THOMAS FLIERL, Nabburg (DE); MARTIN BRABEC, Nabburg (DE)

(21) Appl. No.: 19/186,035

(22) Filed: Apr. 22, 2025

(30) Foreign Application Priority Data
Jul. 1, 2024 (DE) 102024118525.2

(51) Int. Cl. G01N 21/85; G01N 21/94; G01N 27/22; G01N 27/24; G01N 33/18

(52) U.S. Cl. CPC G01N 21/85; G01N 21/94; G01N 27/226; G01N 27/24; G01N 33/1893

(57) ABSTRACT

A sensor apparatus for a water-using household appliance for detecting at least one process parameter includes a housing in which at least one circuit board, a first sensor arrangement and a second sensor arrangement are arranged. The first sensor arrangement is arranged on the at least one circuit board. The second sensor arrangement is spaced apart from the first sensor arrangement along a height axis. A holder element is arranged on the at least one circuit board, on or in which at least one pin-like contact element is arranged. The at least one pin-like contact element is intended and suitable for providing an electrical connection between the second sensor arrangement and the at least one circuit board.

### CROSS-REFERENCE TO RELATED APPLICATIONS

This application claims priority to German Patent Application 102024118525.2, filed on Jul. 1, 2024, the contents of which are incorporated by reference herein in their entirety.

### TECHNICAL FIELD

The invention relates to a sensor apparatus, in particular for a water-using household appliance for detecting at least one process parameter, comprising a housing in which at least one circuit board, a first sensor arrangement and a second sensor arrangement are arranged.

### BACKGROUND

An example of a water-using household appliance is a washing machine. The water-using household appliance comprises a process chamber in which the items to be washed are stored. A process liquid, which is usually water, is supplied to this process chamber. Additives may also be added to the process liquid. Such additives can be, for example, detergents, fabric softeners or similar. In a process, for example a washing process, it is now desirable to monitor certain process parameters. One such process parameter is, for example, the turbidity of the process liquid or the presence of additives in the process liquid. By monitoring process parameters, certain process operations, such as a washing process, can be carried out more effectively and automatically. Corresponding sensor apparatus comprise various sensors. This poses the challenge of positioning the corresponding sensors in the housing in such a way that interference-free detection is guaranteed.

### SUMMARY OF THE INVENTION

The object of the present invention is to provide a sensor apparatus, in particular for a water-using household appliance, which overcomes the above-mentioned disadvantages. Furthermore, it is the object of the present invention to provide a water-using household appliance with such a sensor apparatus.

According to the invention, a sensor apparatus is provided, in particular for a water-using household appliance for detecting at least one process parameter of a water-using household appliance, comprising a housing in which at least one circuit board, a first sensor arrangement and a second sensor arrangement are arranged, the first sensor arrangement being arranged on the at least one circuit board, wherein the second sensor arrangement is spaced along a height axis Z from the first sensor arrangement, wherein a holder element is arranged on the at least one circuit board, on or in which at least one pin-like contact element is arranged, wherein the at least one pin-like contact element is intended and suitable for providing an electrical connection between the second sensor arrangement and the at least one circuit board.

Preferably, the water-using household appliance is a washing machine or a dishwasher. The water-using household appliance advantageously comprises a process chamber in which the items to be washed are stored. A process liquid, which is usually water, is supplied to this process chamber. Advantageously, additives can also be added to the process liquid. Such additives can be, for example, detergents, fabric softeners or similar. One process parameter can be, for example, the turbidity of the process liquid. Turbidity of the process liquid is caused, for example, by dirt particles being washed out of or from the items to be washed. By monitoring a turbidity level, a washing process can be carried out automatically depending on the turbidity of the process liquid or the degree of contamination of the process liquid. The presence of additives in the process liquid can also be a process parameter. It is often desirable for the items to be washed to have no residues of additives, such as detergent, after the process. Detection of such additives in the process liquid can therefore be integrated into an automated washing process. It is also conceivable that a process parameter could be a colour change in the process liquid. In the context of the invention, a colour change is understood to be a deviation in the colour of the process liquid.

The sensor apparatus extends along a height axis (Z), a longitudinal axis (X) and a width axis (Y). By arranging the first sensor arrangement along the height axis (Z) above the second sensor arrangement, a particularly efficient sensor arrangement is provided. Both the first sensor arrangement and the second sensor arrangement can thus perform a corresponding detection without influencing the other sensor arrangement. By providing the pin-like contact element, the second sensor arrangement can also be provided with the required electrical connection to the circuit board.

According to a preferred embodiment, the first sensor arrangement comprises at least one first sensor device and at least one second sensor device. The at least one first sensor device and at least one second sensor device are spaced apart along a width axis (Y). A measuring space is defined between the at least one first sensor device and at least one second sensor device. Such a measuring space is preferably a volume which is filled by the process liquid or through which the process liquid flows. In this measuring space, a measurement of the desired process parameter is thus advantageously carried out on a part of the process liquid. Preferably, the first sensor device and the second sensor device are arranged on the circuit board.

According to a further preferred embodiment, the at least one first sensor device is a radiation source device. Advantageously, the radiation source device emits at least one electromagnetic radiation into the measuring space. The wavelength of the radiation can be in the visible range at 390 nm to 790 nm and or at 780 nm to 3 μm, i.e. in the infrared or near-infrared range. It is also conceivable that the radiation source equipment emits several, preferably several different electromagnetic radiations. For this purpose, the radiation source devices can comprise several radiation sources. The radiation source device preferably emits radiation with a wavelength range that corresponds to a Gaussian or Lorentz distribution around a peak wavelength. Preferably, the at least one second sensor device is a detection device.

According to a further preferred embodiment, the radiation source device can comprise radiation sources which can be selected from the following group: an LED, a laser, a superluminescent LED, a thermal radiator. It would also be conceivable for the radiation source device to comprise different radiation sources and form a combination of the aforementioned list.

According to a further preferred embodiment, the at least one second sensor device is a detection device. Preferably, the detection device detects electromagnetic radiation emerging from the measuring space. This electromagnetic radiation emerging from the measuring space is the transmitted or reflected electromagnetic radiation of the radiation source device. For example, the turbidity of the process fluid can be determined using the transmittance of the electromagnetic radiation.

The detector device can preferably be selected from the group comprising: a phototransistor, a photodiode, a bolometer. Advantageously, the detector devices are broadband detectors that can cover a wide spectral range. It is also conceivable that the detector devices can detect in specific wavelength ranges. It would be conceivable for the detector devices to comprise sensors or active layers in series, for example phototransistors with different band gaps in the active zones. It is also conceivable that 1-pixel or multi-pixel RGB cameras could be used as detector devices. Combinations of the aforementioned components would also be conceivable. Advantageously, the detector devices can comprise further electronic circuits in order to provide a required format of the information signal. Such electronic circuits are then preferably arranged on the circuit board.

According to a further preferred embodiment, the second sensor arrangement comprises at least one third sensor device and at least one fourth sensor device. Preferably, the at least one third sensor device and at least one fourth sensor device are spaced apart along the width axis (Y). Advantageously, the measuring space is defined between the at least one third sensor device and at least one fourth sensor device. The third sensor device and the fourth sensor device are spaced apart from the first sensor device and the second sensor device along the height axis (Z).

According to a further advantageous embodiment, the second sensor arrangement is set up and intended to determine an electrical process parameter of a process fluid in the measuring space. Advantageously, the at least one third sensor device and the at least one fourth sensor device are designed as electrodes. The electrical process parameter is preferably an electrical conductance and/or an electrical capacitance.

The process liquid is therefore located in the measuring space between the third sensor device, which is designed as an electrode, and the fourth sensor device, which is designed as an electrode. The water-based process liquid is generally electrically conductive. By applying a voltage to the electrodes, a conductance or resistance of the process liquid between the electrodes can be determined. This conductance depends on any additives present in the process liquid. If the conductance of the process liquid without additives (i.e. water) is known, it is possible to determine whether the process liquid contains any additives. If this is the case, a further washing process can be initiated, for example.

According to a further preferred embodiment, the third sensor device and the fourth sensor device are intended and set up to directly contact the process fluid in the measuring space. Preferably, the third sensor device and the fourth sensor device are essentially rod-shaped.

The arrangement of the second sensor arrangement according to the invention along the height axis (Z) above the first sensor arrangement thus allows both at least one electrical process parameter and one process parameter, which can preferably be determined using optical sensor devices, to be determined. This arrangement means that the first sensor device and the second sensor device can advantageously be arranged opposite each other. Advantageously, light guides and/or other optical components can thus be dispensed with or the number of such optical components can be significantly reduced. This provides a very simple arrangement, which can be produced more cost-effectively and compactly, as optical components and possibly light guides are not required or are required in smaller dimensions.

According to a further preferred embodiment, the at least one pin-like contact element is arranged on the circuit board. In particular, the pin-like contact element is attached to the circuit board. This can be done, for example, by means of a soldered connection. Advantageously, the pin-like contact element extends from the circuit board along the vertical axis (Z). Preferably, the at least one third sensor device and the at least one fourth sensor device are each assigned at least one pin-like contact element. Accordingly, the number of sensor devices of the second sensor arrangement corresponds to the number of pin-type contact elements. Preferably, therefore, two pin-type contact elements are provided. Preferably, a first pin-type contact element has an electrically conductive connection to the third sensor device and a second pin-type contact element has an electrically conductive connection to the fourth sensor device.

According to a further preferred embodiment, a second contact element is arranged between a pin-like contact element and a sensor device of the second sensor arrangement. The second contact element provides an electrical connection between the respective pin-like contact element and the respective sensor device. Preferably, the second contact element is designed as an elastic element. Preferably, the elastic element consists of an electrically conductive material. Preferably, the elastic element is a spring element. The spring element can be a spiral spring, a torsion spring or similar.

Preferably, the elastic element is arranged with a preload between the pin-like contact element and the respective sensor device of the second sensor arrangement. This pretension ensures a corresponding contact and thus the electrical connection between the pin-like contact element and the respective sensor device. Furthermore, the provision of an elastic element has the advantage that changes in the size of the sensor device, for example an expansion or a reduction in size, are compensated for by a change in the preload. Such changes in size can occur, for example, as a result of thermal influences due to the direct contact of the sensor device with the process fluid. Preferably, the sensor devices of the second sensor arrangement are made of a metal that has a corresponding coefficient of thermal expansion. Conventional electrical connections can be destroyed by such thermal expansion over time. The provision of an electrically conductive elastic element therefore ensures a long-lasting electrical connection.

According to a further advantageous embodiment, the housing has an upper section in which two tower-like elements are formed. Preferably, the tower-like elements are arranged opposite each other along the width axis (Y). Advantageously, the two tower-like elements and the upper section are formed as a single piece or in one piece. Preferably, the at least one first sensor device and at least one second sensor device are each arranged in one of the tower-like elements. Preferably, the circuit board has two finger-like sections which extend along a longitudinal direction (X). Preferably, one finger-like section is arranged in each tower-like element. The measuring space, in which the process fluid is located or through which the process fluid flows, is thus advantageously located between the two tower-like elements.

Preferably, the at least one first sensor device is arranged on one of the finger-like sections of the circuit board and the at least one second sensor device is arranged on the other finger-like section of the circuit board. The two sensor devices are thus arranged opposite each other.

Preferably, the upper section of the housing, but at least the tower-like sections, is made of a material which is essentially transparent to the electromagnetic radiation of the at least one radiation source device. The term “essentially transparent” is intended to express that a passage through the housing does not cause any significant absorption and/or reflection losses for the corresponding radiation.

The upper section of the housing is also preferably designed in such a way that it is sealed against the liquid. Preferably, the housing is made of one or more plastics. Preferably, the housing is manufactured using a plastic injection moulding process.

According to a further advantageous embodiment, the tower-like elements each have a receptacle for the arrangement of a sensor device of the second sensor arrangement. Preferably, the third sensor device and the fourth sensor device are each arranged in a receptacle of a tower-like element. Preferably, the third sensor device and the fourth sensor device each extend through an opening in the upper section of the housing. It is advantageous that the third sensor device and the fourth sensor device each have a sealing element, which is arranged between the respective sensor device and the respective opening. This means that the third sensor device and the fourth sensor device are in direct contact with the process fluid. The interior of the housing is protected by the seal with the respective sealing element.

According to a further advantageous embodiment, a lower section of the housing adjoins the upper section of the housing. A sealing element is preferably arranged in an upper area of the lower section of the housing on an outer wall of the lower section. Advantageously, the sealing element is designed to completely encircle the circumference of the lower section. The sensor apparatus advantageously protrudes into the process chamber of the household appliance. The upper section is preferably arranged in the process chamber. The lower section is preferably intended and suitable for enabling a signalling connection with the household appliance, in particular with a control device of the household appliance. Advantageously, the lower section has an opening through which a plug element can be inserted into the housing. The arrangement of the sensor device in the process chamber is sealed by the sealing element.

Preferably, a control device is provided, which is connected to the first sensor arrangement and the second sensor arrangement in terms of signalling. Advantageously, the control device is associated with the household appliance. However, it would also be conceivable for a control device to be associated with the sensor device. Such a control device associated with the sensor device could then be signalled to a control device of the household appliance.

According to a further advantageous embodiment, the holder element has a latching section which can be latched onto a plug element that can be arranged on the circuit board. Such a plug element is advantageously inserted through the opening of the lower section of the housing. Preferably, the circuit board has contact surfaces which come into contact with the plug element. Preferably, the plug element is pushed onto the circuit board and thus advantageously engages around a part of the circuit board that comprises the corresponding contact surfaces. The latching section provides a detachable positive connection with the plug element. The plug element thus has a secure hold on the circuit board.

According to a further advantageous embodiment, the holder element has at least one arm section. Preferably, the arm section has a fastening element. Preferably, the fastening element attaches the arm section to the circuit board. For example, a form-fit connection between the fastening element and the circuit board can be provided for this purpose. Other, possibly detachable connections, such as snap-on connections, are also conceivable. Preferably, the fastening element has a hook-like design and engages through a hole in the circuit board. It is also conceivable that the fastening element is designed as a recess or a hole in the arm element, which receives a complementary fastening element from the circuit board. The arm section with the fastening element ensures appropriate mechanical strength. This means that any forces that occur when the latching section of the retaining element engages or disengages from the connector element do not affect the retaining element.

The problem is further solved by a household appliance comprising at least one sensor apparatus according to one of the embodiments described above. The household appliance can be equipped with all the features already described above in the context of the sensor apparatus, either individually or in combination with one another, and vice versa.

Advantageously, the household appliance is a water-using household appliance, for example a washing machine or a dishwasher.

### BRIEF DESCRIPTION OF DRAWINGS

Further advantages, objectives and features of the present invention are explained with reference to the following descriptions of the attached figures. Similar components may have the same reference signs in the various embodiments.

It shows:

FIG. 1 illustrates a household appliance with a sensor apparatus;

FIG. 2 illustrates a sensor apparatus according to one embodiment;

FIG. 3 is a sectional view of a sensor apparatus according to one embodiment;

FIG. 4 illustrates a part of a sensor apparatus according to one embodiment;

FIG. 5 illustrates a part of a sensor apparatus according to one embodiment;

FIG. 6 illustrates a retaining element according to one embodiment and

FIG. 7 illustrates a sensor apparatuses according to one embodiment.

In the figures, identical components are to be understood with the corresponding reference signs. For the sake of clarity, components may not be labelled with a reference symbol in some figures, but have been designated elsewhere.

### DETAILED DESCRIPTION

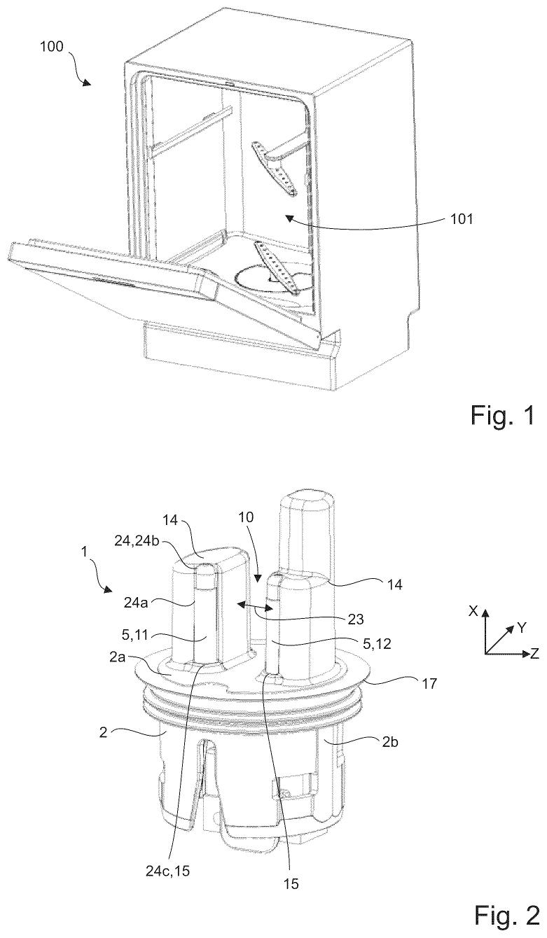

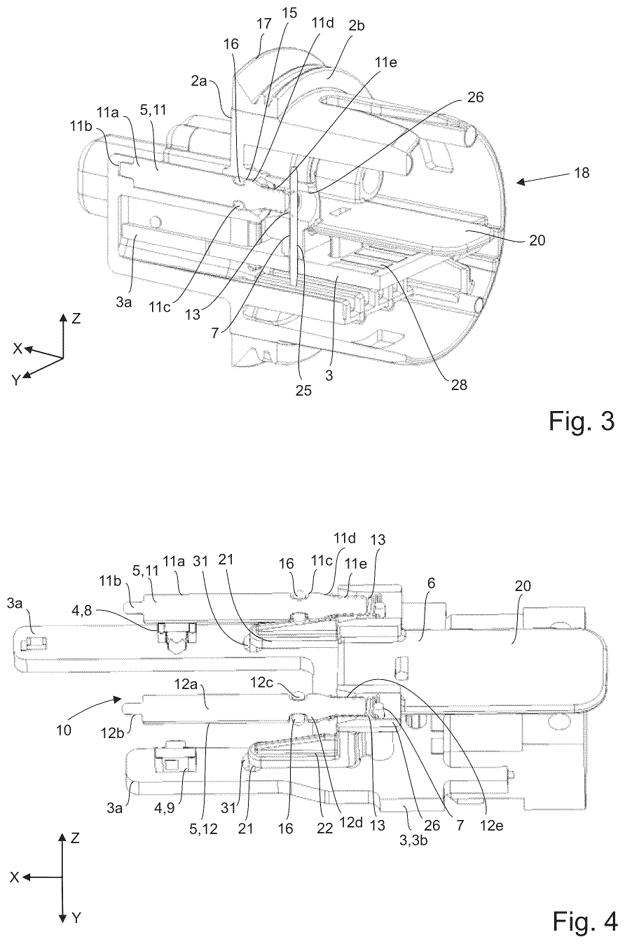

FIGS. 2 to 6 show a sensor apparatus 1, in particular for a water-using household appliance 100 for detecting at least one process parameter. The sensor apparatus 1 comprises a housing 2, in which at least one circuit board 3, a first sensor arrangement 4 and a second sensor arrangement 5 are arranged. The first sensor arrangement 4 is arranged on the at least one circuit board 3, wherein the second sensor arrangement 5 is spaced apart from the first sensor arrangement 4 along a height axis Z, wherein a holder element 6 is arranged on the at least one circuit board 3, on or in which at least one pin-like contact element 7 is arranged, wherein the at least one pin-like contact element 7 is intended and set up to provide an electrical connection between the second sensor arrangement 5 and the at least one circuit board 3.

FIG. 1 shows a water-using household appliance 100 comprising a process chamber 101 into which a process fluid can be introduced. The household appliance 100 can, for example, be designed as a dishwasher, a washing machine or the like. The sensor apparatus 1 is arranged in the household appliance 1. It is preferably provided that the sensor apparatus 1 is at least partially in contact with the process liquid.

FIG. 2 shows sensor apparatus 1, which comprises an exemplary housing 2. The housing 2 comprises two tower-like elements 14, which are spaced apart from each other by a distance 23. Between the tower-like sections 14 there is an intermediate area, which is defined as the measuring space 10. Process fluid is present in the measuring space 10 during a process, for example a washing process, or the process fluid is flushed through the measuring space 10 during the process.

The housing 2 comprises an upper section 2a and a lower section 2b. The upper section 2a merges integrally into the lower section. In particular, the housing 2, i.e. the upper section 2a and the lower section 2b, is formed integrally or in one piece. The tower-like elements 14 are formed in integrally or in one piece with the upper section 2a, whereby a integrally design means that all sections are manufactured from a single and uniform part. A one-piece design means that all sections are not manufactured from a single and uniform part, but are not only firmly connected to each other, but are so intimately connected that they do not appear as several components joined together and in any case can no longer be detached from each other without destroying them in the process. The housing 2 is made of at least one plastic material.

Advantageously, the housing 2 protrudes partially into the process chamber 101. For this purpose, an opening is provided in a wall of the process chamber 101. The lower section 2b is essentially designed as a circular hollow cylinder. A sealing element 17 is arranged in an upper area of the lower section 2b. This sealing element 17 has at least one sealing lip. This sealing element 17 ensures a tight closure of the opening in the process chamber 101. The upper section 2a with the two tower-like elements 14 is therefore located inside the process chamber 101 and the lower section 2b is located outside the process chamber 101.

The first sensor arrangement 4 and a second sensor arrangement 5 are provided for detecting the process parameters. Of course, it is also conceivable that further sensor arrangements could be provided.

The first sensor arrangement 4 is an optical sensor arrangement and comprises at least one first sensor device 8 in the form of a radiation source device and at least one second sensor device 9 in the form of a detection device. The at least one first sensor device 8 in the form of a radiation source device emits at least one electromagnetic radiation into the measuring space 10. It is also conceivable that several electromagnetic radiations with different wavelength ranges are emitted into the measuring space 10. The electromagnetic radiation can comprise wavelengths in the visible range at 390 nm to 790 nm and or at 780 nm and 3 μm, i.e. in the infrared or near-infrared range. The at least one electromagnetic radiation from the radiation source devices passes through the process fluid in the measuring space 10 (transmission geometry) and/or is reflected in the measuring space (reflection geometry). The electromagnetic radiation emitted from the measuring space 10 is then detected by the at least one second sensor device 9 in the form of a detection device. Particularly for transmission measurement, it is advantageous that the at least one first sensor device 8 in the form of a radiation source device and the at least one second sensor device 9 in the form of a detection device are arranged opposite each other along the width axis Y.

The at least one first sensor device 8 and the at least one second sensor device 9 are each arranged in one of the tower-like elements 14. Furthermore, the at least one first sensor device 8 and the at least one second sensor device 9 are arranged on the circuit board 3. For this purpose, the circuit board 2 comprises finger-like sections 3a, which extend along the longitudinal axis X. Each of the finger-like sections 3a extends into one of the tower-like elements 14. Furthermore, the finger-like sections 3a open into a main section 3b of the circuit board 3.

The tower-like elements 14 are thus intended to protect the optical components from direct contact with the process fluid. The tower-like elements 14, possibly the entire upper section 2a, are made of a material that is essentially transparent to the above-mentioned electromagnetic radiation. The lower section 2b of the housing 2 does not necessarily have to be transparent to the respective radiation, but it can be.

The second sensor arrangement 5 comprises at least one third sensor device 11 and at least one fourth sensor device 12. The at least one third sensor device 11 and the at least one fourth sensor device 12 are designed as electrodes. Accordingly, the second sensor arrangement 5 is set up and intended to determine an electrical process parameter of a process fluid located in the measuring space 10. The electrical process parameter is an electrical conductance and/or an electrical capacitance. Such conductance measurements are used, for example, to determine whether and to what degree the process fluid contains an additive.

A voltage is applied between the two electrodes to detect the conductance. For this purpose, it is necessary for the third sensor device 11 and the fourth sensor device 12 to be in direct contact with the process liquid.

FIGS. 2, 3, 4 and 5 show that the third sensor device 11 and the fourth sensor device 12 are essentially rod-shaped. The third sensor device 11 and the fourth sensor device 12 each extend along the height axis Z above one of the finger-like sections 3a of the circuit board 3 and are thus each located along the height axis Z above one of the sensor devices 8, 9 of the first sensor arrangement 4. According to the embodiment shown in the figures, the third sensor device 11 is thus located above the first sensor device 8. The fourth sensor device 12 is located above the second sensor device 9. Thus, the third sensor device 11 and the first sensor device 8 are spaced along the width axis Y from the fourth sensor device 12 of the second sensor device 9.

The two sensor devices 11, 12 of the second sensor arrangement 5 have a main section 11a, 12a, which is essentially circular-cylindrical in shape. However, other geometric shapes would also be conceivable, for example cuboid, etc. At one front end of the two sensor devices 11, 12, a fastening section 11b, 12b adjoins the main section 11a, 12a. The fastening section 11b, 12b is pin-shaped and has a smaller diameter than the main section 11a, 12a.

Opposite the fastening section 11b, 12b, the main section 11a, 12a merges into a receiving section 11c, 12c. The receiving section 11c, 12c, which accommodates a sealing element 16 in the form of an O-ring, is trough-shaped and therefore has a smaller diameter than the main section 11a, 12a. A transition section 11d, 12d is provided downstream of the receiving section 11c, 12c. This transition section 11d, 12d initially has a diameter that essentially corresponds to the diameter of the main section 11a, 12a. As it continues along the longitudinal axis X towards a rear end of the respective sensor device 11, 12, the diameter of the transition section 11d, 12d decreases continuously. The transition section 11d, 12d finally opens into an end section 11e, 12e, which has a smaller diameter than the main section 11a, 12a.

The tower-like elements 14 each have a receptacle 24, in each of which a sensor device 11, 12 of the second sensor arrangement 5 is accommodated. The receptacle 24 has a first section 24a, in which the main section 11a, 12a of the respective sensor device 11, 12 is arranged. This first section 24a has a circular arc-like cross-section, so that a part of the lateral surface of the main section 11a, 12a is in contact with the first section 24a of the receptacle 24. The remaining part of the lateral surface is thus exposed and can contact the process fluid. Furthermore, the receptacle 24 has a second section 24b, in which the pin-like end section 11b, 12b is accommodated. The end section 11b, 12b is completely surrounded by the second section 24b.

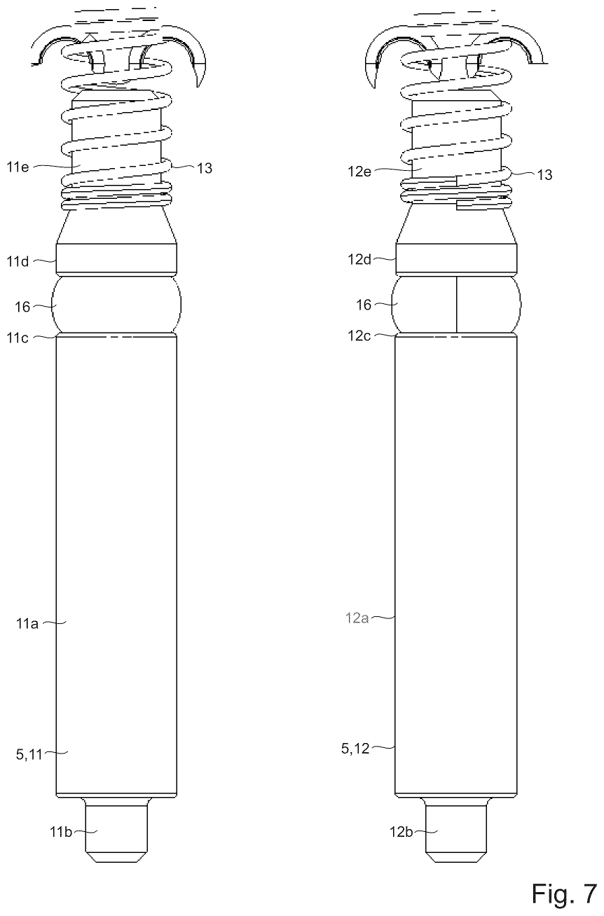

The third sensor device 11 and the fourth sensor device 12 each extend through an opening 15 in the upper section 2a of the housing 2; this opening 15 is provided in a third section 24c receptacle 24. The third section 24c is designed as a wooden cylinder-like bearing element with a circular cross-section. The sealing element 16, which is designed as an O-ring and is arranged in the receiving section 11c, 12c of the sensor devices 11, 12, rests against an inner wall of the third section 24c, thereby ensuring a seal with respect to the interior of the lower section 2b of the housing 2. The respective sensor device 11, 12 is thus held by the first section 24a and the third section 24c of the respective receptacle 24. The third sensor device 11 and the fourth sensor device 12 are shown in more detail in FIG. 7.

The at least one pin-like contact element 7 is arranged on the circuit board 3 and extends from the circuit board 3 along the height axis Z. The electrical connection between the pin-like contact element 7 and the circuit board 3 is made via a soldered connection, for example. The at least one third sensor device 11 and the at least one fourth sensor device 12 are each assigned at least one pin-like contact element 7. According to the embodiments shown in the figures, two pin-type contact elements 7 are thus provided.

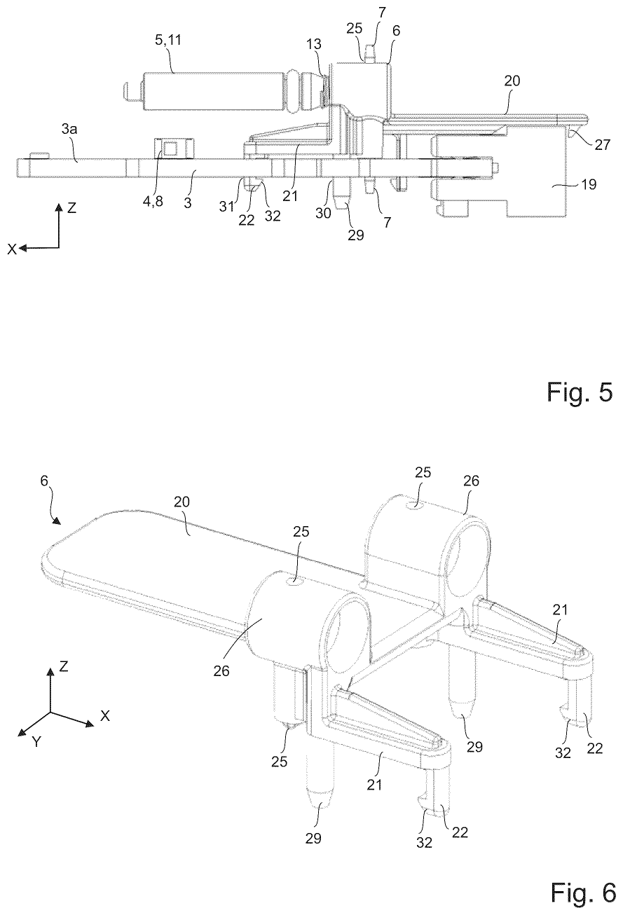

The pin-like contact elements 7 are also held by the holder element 6. A first through channel 25 is provided in the holder element 6 for this purpose. According to FIG. 5, the contact element 7 protrudes both beyond the holder element 6, or out of the first through channel 25, and beyond the circuit board 3.

In each case, a second contact element 13 is arranged between a pin-like contact element 7 and a sensor device 11, 12 of the second sensor arrangement 5. The second contact element 13 provides an electrical connection between the respective pin-like contact element 7 and the respective sensor device 11, 12. The second contact element 13 is designed as an elastic element, in particular a spring element. The second contact element 13 is partially arranged on the end section 11e, 12e of the respective sensor device 11, 12. Furthermore, the second contact element 13 is in contact with the pin-like contact element 7, so that there is an electrical connection between the pin-like contact element 7 and the respective sensor device 11, 12.

Furthermore, the second contact element 13, which in the present case consists of a spiral spring element, provides a preload between the pin-like contact element 7 and the respective sensor device 11, 12. The preload is caused by the fact that the pin-like contact element 7 is fastened by the holder element 6 and the respective sensor device 11, 12 is fastened by the receptacle 24. Since the sensor devices 11, 12 are preferably made of a metal, a change in temperature of the process fluid can cause thermal expansion of the respective sensor device 11, 12. Such thermal expansion can be compensated for by the second contact element 13.

Only the preload changes. A mechanically fixed conductive connection would be damaged by such thermal expansions.

The holder element 6 has two hollow cylinder-like second receptacles 26, which have a circular cross-section. The second receptacles 26 comprise a second lead-through 26a, which extends along the longitudinal axis X. The respective end section 11b, 12b of the respective sensor element 11, 12 protrudes into these second receptacles 26, or the second lead-throughs 26a. The respective first through-channel 25, in which the pin-like contact element is arranged, runs vertically, i.e. essentially parallel to the height axis Z to the respective second through-channel 26.

FIG. 6 shows the holder element 6. In addition to the components already described above, the holder element 6 also has a latching section 20, which can be latched onto a plug element 19 that can be arranged on the circuit board 3. The latching section 20 is plate-like and has a latching projection 27 on a lower side, which can engage with the plug element 19. The latching section 20 has sufficient elasticity so that it can be deformed to release the latching connection to the plug element 19 or to raise the latching projection sufficiently far along the height axis Z.

The plug element 19 embraces a section of the circuit board 3. The circuit board 3 has corresponding contact surfaces 28, which are in contact with the plug element 19. The plug element 19 can be used to establish a wired connection to the household appliance 100 or a control device of the household appliance 100

For mechanical fastening of the retaining element 6 to the circuit board 3, the retaining element 6 comprises two pin-like elements 29, which are arranged substantially along the height axis Z below the second receptacles 26. These pin-like elements 29 protrude through corresponding first holes 30 of the circuit board 3.

Furthermore, the holder element 6 has at least one arm section 21. In the present case, two arm sections 21 are provided. The arm section 21 has a fastening element 22, which is intended to fasten the arm section 21 and thus the holder element 6 to the circuit board 3. The fastening element 22 has a pin-like section, at the end of which a hook-like projection 32 is arranged. The fastening elements 22 protrude through second holes 31. The hook-like projections 32 then engage on an underside of the circuit board 3.

It would be conceivable to provide an electronic circuit that converts the electrical signal from the detection device into the information signals required by the control device. Corresponding information signals can be an electrical current, an electrical voltage, a PWM (pulse width modulated) signal or an AM (amplitude modulated) signal. This electronic circuit is preferably arranged on the circuit board. The circuit board 3 is, for example, a PCB (printed circuit board).

The control unit can initiate appropriate measures based on the measured process parameters. This can be, for example, an adjustment to the process, such as a washing process. Furthermore, a corresponding output signal could also be issued for a user. For this purpose, the control device can be connected to an output device and/or a communication device using signalling. The output signal can be a visual or acoustic signal, for example. The communication device can be used, for example, to send a message to a user's device, such as a smartphone, laptop or similar.

The applicant reserves the right to claim all features disclosed in the application documents as being essential to the invention, provided that they are new, either individually or in combination, compared with the prior art. It should also be noted that the individual figures also describe features which may be advantageous in themselves. The person skilled in the art immediately recognises that a certain feature described in a figure can also be advantageous without the adoption of further features from this figure. Furthermore, the skilled person recognises that advantages can also result from a combination of several features shown in individual figures or in different figures.

### LIST OF REFERENCE SYMBOLS

1 Sensor apparatus
2 Housing
2a Upper section of the housing
2b Lower section of the housing
3 Circuit board
3a Finger-like sections of the circuit board
3b Main section of the circuit board
4 First sensor arrangement
5 Second sensor arrangement
6 Holder element
7 Pin-type contact element
8 First sensor device
9 Second sensor device
10 Measuring space
11 Third sensor device
11a Main section
11b Fastening section
11c Receiving section
11d Transition section
11e End section
12 Fourth sensor device
12a Main section
12b Fastening section
12c Receiving section
12d Transition section
12e End section
13 Second contact element
14 Tower-like elements
14a Receptacle
15 Opening in housing
16 Sealing element
17 Sealing element
18 Opening
19 Plug element
20 Latching section of the holder element
21 Arm section
22 Fastening element
23 Distance
24 Receptacle
24a First section of the receptacle
24b Second section of the receptacle
24c Third section of the receptacle
25 Lead-through
26 Second receptacle
26a Second lead-through
27 Latching protrusion
28 Contact surfaces
29 Pin-like elements
30 First hole of the circuit board
31 Second hole of the circuit board
32 Hook-like projections
100 Household appliance
101 Process chamber
X Longitudinal axis
Y Width axis
Z Height axis

1. Sensor apparatus (1) for a water-using household appliance (100) for detecting at least one process parameter, comprising:
a housing (2) in which at least one circuit board (3), a first sensor arrangement (4) and a second sensor arrangement (5) are arranged,
wherein
the first sensor arrangement (4) is arranged on the at least one circuit board (3), wherein the second sensor arrangement (5) is spaced apart from the first sensor arrangement (4) along a height axis (Z), wherein a holder element (6) is arranged on the at least one circuit board (3), on or in which at least one pin-like contact element (7) is arranged, wherein the at least one pin-like contact element (7) is intended and suitable for providing an electrical connection between the second sensor arrangement (5) and the at least one circuit board (3).

2. Sensor apparatus (1) according to claim 1, wherein
the first sensor arrangement (4) comprises at least one first sensor device (8) and at least one second sensor device (9), wherein the at least one first sensor device (8) and at least one second sensor device (9) are spaced apart along a width axis (Y), wherein a measuring space (10) is defined between the at least one first sensor device (8) and at least one second sensor device (9).

3. Sensor apparatus (1) according to claim 2, wherein
the at least one first sensor device (8) is a radiation source device, wherein the at least one second sensor device (9) is a detection device, wherein the radiation source device emits at least one electromagnetic radiation into the measuring space (10), wherein the detection device detects an electromagnetic radiation emerging from the measuring space (10).

4. Sensor apparatus (1) according to claim 1, wherein
the second sensor arrangement (5) comprises at least one third sensor device (11) and at least one fourth sensor device (12), wherein the at least one third sensor device (11) and at least one fourth sensor device (12) are spaced apart along the width axis (Y), wherein the measuring space (10) is defined between the at least one first sensor device (11) and at least one second sensor device (12).

5. Sensor apparatus (1) according to claim 4, wherein
the second sensor arrangement (5) is set up and intended to determine an electrical process parameter of a process fluid located in the measuring space (10), wherein the at least one third sensor device (11) and the at least one fourth sensor device (12) are designed as electrodes, wherein the electrical process parameter is an electrical conductance and/or an electrical capacitance.

6. Sensor apparatus (1) according to claim 4, wherein
the third sensor device (11) and the fourth sensor device (12) are intended and set up to directly contact the process liquid in the measuring space (10), wherein the third sensor device (11) and the fourth sensor device (12) are substantially rod-like.

7. Sensor apparatus (1) according to claim 4, wherein
the at least one pin-like contact element (7) is arranged on the circuit board (3) and extends from the circuit board (3) along the height axis (Z), wherein the at least one third sensor device (11) and the at least one fourth sensor device (12) are each assigned at least one pin-like contact element (7).

8. Sensor apparatus (1) according to claim 7 further comprising
a second contact element (13) arranged in each case between a pin-like contact element (7) and a sensor device (11, 12) of the second sensor arrangement (5), wherein the second contact element (13) provides an electrical connection between the respective pin-like contact element (7) and the respective sensor device (11, 12), wherein the second contact element (13) acts as an elastic element, wherein the elastic element is a spring element.

9. Sensor apparatus (1) according to claim 2, wherein
the housing (2) has an upper section (2a) in which tower-like elements (14) are arranged, wherein the tower-like elements (14) are arranged opposite one another along the width axis (Y), wherein the at least one first sensor device (8) and at least one second sensor device (9) are each arranged in one of the tower-like elements (14), wherein the circuit board (3) has two finger-like sections (3a) which extend along a longitudinal direction (X), wherein in each case a finger-like section (3a) is arranged in a tower-like element (14).

10. Sensor apparatus (1) according to claim 9, wherein
the tower-like elements (14) each have a receptacle (24), wherein the third sensor device and the fourth sensor device are each arranged in a receptacle (14a), wherein the third sensor device (11) and the fourth sensor device (12) each extend through a opening (15) in the upper section (2a) of the housing (2), wherein the third sensor device (11) and the fourth sensor device (12) each have a sealing element (16) which is arranged between the respective sensor device (11, 12) and the respective opening (15).

11. Sensor apparatus (1) according to claim 9, further comprising
a lower section (2b) of the housing (2) adjoining the upper section (2a) of the housing (2), wherein a sealing element (17) is arranged on an outer wall of the lower section (2b) in an upper region of the lower section (2b) of the housing (2), wherein the lower section (2b) has an opening (18) through which a plug element (19) can be inserted into the housing (2).

12. Sensor apparatus (1) according to claim 1, wherein
the holder element (6) has a latching section (20), which can be latched into a plug element (19) that can be arranged on the circuit board (3).

13. Sensor apparatus (1) according to claim 1, wherein
the holder element (6) has at least one arm section (21), wherein the at least one arm section (21) has a fastening element (22) which fastens the arm section (21) to the circuit board (3).

14. Household appliance (1) comprising at least one sensor device according to claim 1.

15. Sensor apparatus (1) for a water-using household appliance (100) for detecting at least one process parameter, comprising:
a housing (2);
at least one circuit board (3), a first sensor arrangement (4), and a second sensor arrangement (5) arranged in the housing (2), wherein the first sensor arrangement (4) is arranged on the at least one circuit board (3) and the second sensor arrangement (5) is spaced from the first sensor arrangement (4) along a height axis (Z);
a holder element (6) arranged on the at least one circuit board (3); and
at least one pin-like contact element (7) arranged on or in the holder element (6) and configured to provide an electrical connection between the second sensor arrangement (5) and the at least one circuit board (3).
